(19) United States
(12) Patent Application Publication
(10) Pub. No.: US 2009/0264969 A1
(43) Pub. Date: Oct. 22, 2009

(54) Multi-mode cooling garment

(75) Inventor: Clifford E. Gammons, Loudon, TN (US)

Correspondence Address: KNOX PATENTS, P.O. BOX 30034, KNOXVILLE, TN, 37930-0034, US

(73) Assignee: ADROIT DEVELOPMENT, INC., Loudon, TN (US)

(21) Appl. No.: 12/362,266

(22) Filed: Jan. 29, 2009

Related U.S. Application Data
(60) Provisional application No. 61/046,429, filed on Apr. 20, 2008.

(51) Int. Cl. A61F 7/00

(52) U.S. Cl. 607/104; 607/108; 607/114

(57) ABSTRACT

Apparatus for cooling a person in extreme environments. The apparatus includes a garment having a vest with attached tubing. The vest includes an evaporative cooling device and the tubing is configured to circulate chilled water. The vest includes a material that is able to receive and absorb an amount of liquid water with a mass greater than the material. When liquid water is applied to the vest, the vest absorbs the water for later evaporation. The tubing enables the garment to have a lower differential temperature between the inside surface and the outside surface, thereby reducing the rate of evaporation, which enables the passive cooling to be operable for an extended period of time. When the heat load does not permit the differential temperature to be reduced, the tubing removes additional heat, thereby providing additional heat removing capacity beyond the passive cooling.

### CROSS-REFERENCE TO RELATED APPLICATIONS

This application claims the benefit of U.S. Provisional Application No. 61/046,429, filed Apr. 20, 2008.

### STATEMENT REGARDING FEDERALLY SPONSORED RESEARCH OR DEVELOPMENT

Not Applicable

### BACKGROUND OF THE INVENTION

1. Field of Invention

This invention pertains to a garment configured to be worn by a person or animal in a hot environment. More particularly, this invention pertains to a garment with multiple modes of cooling.

2. Description of the Related Art

Many occupations require exposure to severe environments, such as those with extreme temperatures. When the ambient temperature is very cold, an individual can wear insulative layers of clothing, although at the expense of mobility, flexibility, and overall size or bulk. When the ambient temperature is very hot, the individual can remove only so much clothing in order to obtain relief. Often, in such an environment, the individual must wear protective clothing and gear, which cannot be readily or completely removed.

For example, firefighters enter burning buildings that frequently have exposed flames and are filled with smoke. In order to protect themselves, firefighters wear protective clothing, air packs, helmets with face shields, and other gear. Wearing the protective clothing in hot, fire filled buildings leads to heat exhaustion for firefighters and limits the time that the firefighter is available for combating the fire. Athletes also often face overheating conditions, for example, a football player practicing in the summer heat.

It is not uncommon for such persons to combat heat exhaustion by dousing themselves with cool water to lower their body temperature. Water sprays or misting systems also provide a cooling effect through evaporative cooling. Severe cases of heat exhaustion and heatstroke are treated by immersion in a cool water bath or by the application of ice packs to the body.

Another way to combat heat exhaustion is active cooling. For example, Published Patent Application Number 2007/0085340, titled “Upper Body Thermal Device with Quick-Disconnect Connectors,” discloses a garment with tubing attached to the outside of the fabric. The tubing carries conditioned fluid for cooling the body of a wearer. The thermal effect from such a garment is limited by the area covered by the tubing carrying the liquid and the temperature of the liquid.

### BRIEF SUMMARY OF THE INVENTION

According to one embodiment of the invention, a multi-mode cooling garment includes both active cooling and passive cooling features to achieve longer lasting and more rapid cooling of a person or animal than that available from only active cooling and passive cooling. A particular advantage of the garment is that cooling is available for an extended time. The garment includes a vest with tubing attached to the vest. The vest is an evaporative cooling device that receives liquid water and permits the water to evaporate over time. The tubing is part of an active cooling device that circulates conditioned fluid for heat transfer.

In one embodiment, the vest has an inner layer that is water resistant, a middle layer that absorbs liquid water and releases water vapor, and an outer layer that passes water as a liquid and a vapor. The middle layer includes a polymer, such as a superabsorbent polymer or polyacrylamide. The polymer absorbs liquid water and releases it as the water evaporates. The evaporation cools the wearer of the vest. Attached to the vest is tubing that circulates cooled water. The circulating water removes heat from the vest and reduces the temperature differential between the inner and outer layers.

For evaporative cooling, cooling lasts only as long as fluid remains for evaporation, and the amount of fluid is directly related to the size and weight of the evaporative cooling device. For active cooling, cooling lasts only as long as a source of cold producing material is available, and the quantity of cold producing material is directly related to the size and weight of the active cooling device. With both evaporative and active cooling, the garment has reduced bulk and weight compared to an equivalent garment with only evaporative or active cooling. Also, with both evaporative and active cooling, the garment has increased heat transfer capabilities compared to an equivalent garment with only evaporative or active cooling.

The garment is part of a system that includes multiple garments that connect to a water supply. The water supply includes manifolds for connecting the multiple garments to a pump drawing chilled water from a reservoir. In one embodiment, the reservoir contains ice and water. The ice melts from the warm water returned from the tubes on the garment. The melting ice cools the water in the reservoir and the cooled water is circulated to the garment where the water absorbs heat. During the time the water is circulating, the garment is also undergoing evaporative cooling. Before use, the vest is soaked in water to allow the vest to absorb the liquid. In use, the water evaporates. Because the circulating water in the tubes is removing heat, the rate of evaporation is better matched to the heat load on the garment.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view of one embodiment of a multi-mode cooling garment;

FIG. 2 is a front view of the back of one embodiment of the inside of the cooling garment that is turned inside-out;

FIG. 3 is an exploded diagram showing one embodiment of the layers of a garment; and

FIG. 4 is a schematic diagram showing a one embodiment of multi-mode cooling system.

### DETAILED DESCRIPTION OF THE INVENTION

Apparatus for a multi-mode cooling system 400 with multiple cooling garments 100 is disclosed. The system 400 includes garments 100 with multi-mode cooling, that is, the garments 100 include both active cooling and passive cooling features, which results in a cooling system that lasts longer and cools more rapidly than a comparable garment with only active cooling or passive cooling. As used herein, a garment is a covering for a portion of the body, such as an article of clothing like a vest, shirt, or pants.

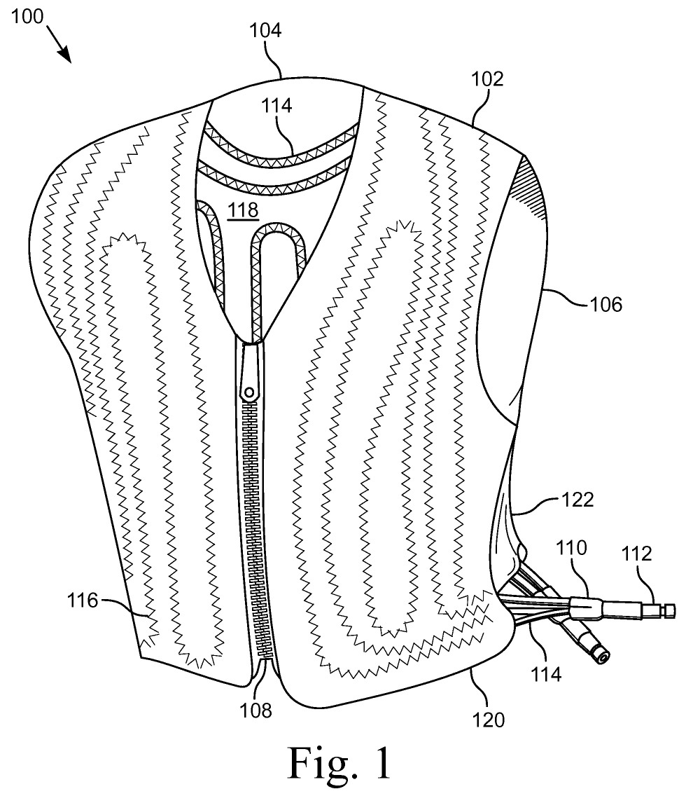

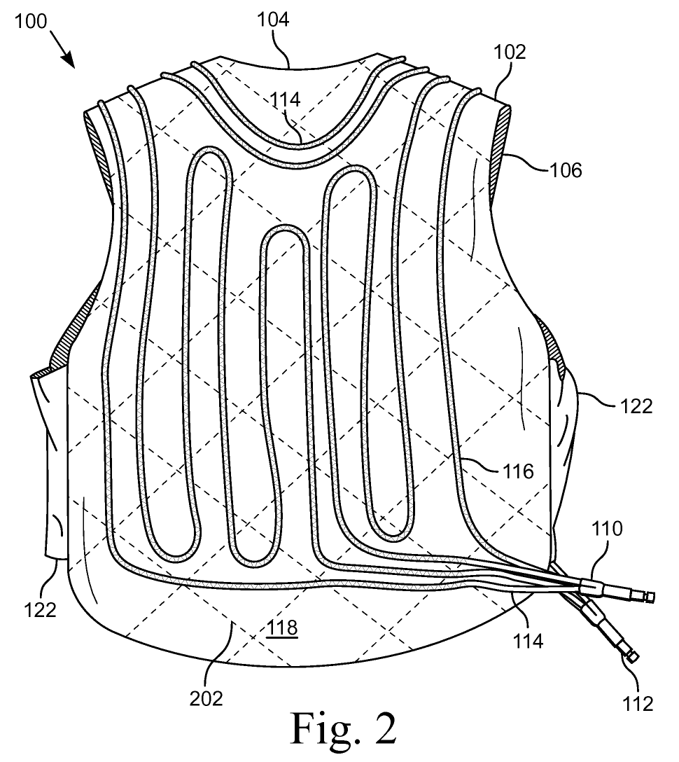

FIG. 1 illustrates a perspective view of one embodiment of a multi-mode cooling garment 100. FIG. 2 illustrates a front view of the back 118 of one embodiment of the inside of the cooling garment 100 that is turned inside-out. The illustrated garment 100 includes a vest 102 and several sections of tubing, or fluid conduit, 114 attached to the vest 102 with thread 116. The garment 100 is an article of clothing configured to fit around a portion of the body of the wearer.

The vest 102 includes a front section 120 and a back section 118. The front section 120 of the vest 102 is in two halves that are releasably connected with a zipper 108. In other embodiments, the two halves of the front section 120 are releasably joined with another type of fastener, for example, a hook and loop fastening system or buttons or snaps. The two sections 118, 120 of the vest 102 are joined at each side with a gusset 122. In one embodiment, the gusset 122 is an elastic material that allows the two sections 118, 120 to better conform to the shape of the wearer's torso. An elastic gusset 122 ensures that the inside of the garment 100 contacts as much of the wearer as possible and prevents the garment 100 from sagging or fitting loosely, which would decrease the cooling effect on the wearer.

The vest 102 includes a material that absorbs liquid water quickly and subsequently allows the water to evaporate. The evaporation of the water transfers heat from the garment 100 and from the wearer. Evaporative cooling provides the passive cooling feature of the garment 100.

In FIG. 2 the garment 100 is turned inside-out and the view illustrates the serpentine arrangement of the tubing 114 on the inside back surface 118 of the garment 100. The material of the inside surface fabric is substantially water resistant such that the fluid retained by the garment 100 does not wick to or wet an object in contact with the inside surface 118. For example, a person wearing a shirt under the garment 100 would feel cooler wearing the garment 100, but the person's shirt would not be dampened or wetted by any fluid from garment 100.

In the illustrated embodiment, the tubing 114 is sewn to the inside surface of the vest 102 with a thread 116. The tubing 114 is arranged in multiple loops with the ends terminated in a manifold 110 with a connector end 112. Each of the manifolds 110 connect the connector end 112 to four ends of tubing 114. The ends of the tubing 114 are illustrated protruding from the side of the vest 102 below a gusset 122. In other embodiments the ends of the tubing 114 protrude for another part of the vest 102 so as to provide a convenient attachment to a source of cooling water. For example, the ends of the tubing 114 exit from the bottom of the back section 118, which is convenient for allowing the connectors 112 to automatically disconnect when the wearer moves forward.

Each loop or run of tubing 114 extends from one manifold 110 to the other manifold 110. The tubing 114 follows a serpentine path on the surface of the vest 102 and the serpentine path is configured to evenly distribute the tubing 114 on the surface of the vest 102 to ensure even heat transfer between the tubing 114 and the wearer of the garment 100 and between the tubing 114 and the vest 102.

A heat transfer or heat exchange area is defined where the tubing, or fluid conduit, 114 is attached to the vest 102. For example, in the illustrated embodiment, the tubing 114 is arranged in a serpentine pattern on the front section 120 and the back section 118 of the vest 102, but not on the sides of the vest 102 where the gussets 122 are located. Such a configuration positions a heat transfer area adjacent the front of the wearer and a heat transfer area adjacent the wearer's back. The sides of the garment 100 do not have a heat transfer area and, subsequently, the sides are less bulky and allow the wearer freedom of movement of the arms. In another embodiment, the fluid conduit 114 is formed as a conduit or fluid pathway in or attached to the vest. For example, the fluid conduit 114 is formed by joining two layers together in all areas except where the fluid conduit 114 is located.

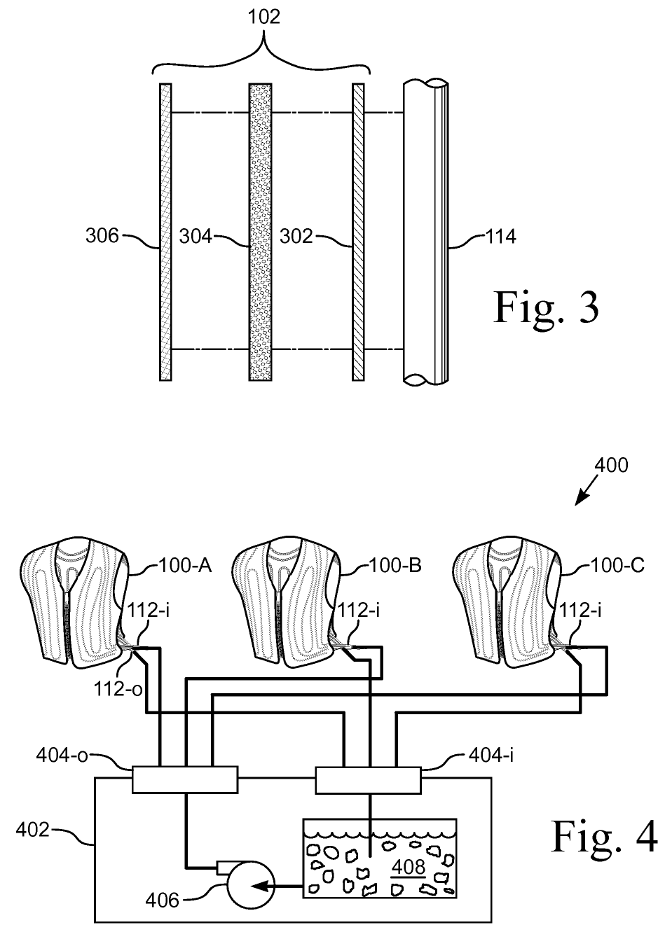

FIG. 3 illustrates an exploded diagram showing one embodiment of the layers of the garment 100. The vest 102 incorporates passive cooling. In the illustrated embodiment, the vest 102 has three layers: an inner layer 302, an outer layer 306, and a middle layer, or filler, 304. The layers 302, 304, 306 form a sandwiched structure, or sheet, with the layers 302, 304, 306 adjacent to each other and contacting each other. In one such embodiment, the layers 302, 304, 306 are joined loosely, such as by quilting or stitching through the layers 302, 304, 306. FIG. 2 illustrates the quilt stitching 202 on the back section 118 of the vest 102. The quilting stitching 202 allow the middle layer 304 to expand in thickness without being compressed between the inner and outer layers 302, 306. The middle layer 304 expands in thickness when the middle layer 304 absorbs the liquid water as the vest 102 is charged with liquid water.

In other embodiments, at least two of the layers 302, 304, 306 are bonded or integrally formed, for example, the inner layer 302 is a skin or treated surface of the middle layer 304. In one such embodiment, the inner layer 302 is a surface on the middle layer 304 and the outer layer 306 is a surface on the middle layer 304. For example, the middle layer 304 is a porous material and the inner layer 302 is a surface of the middle layer 304 that is impermeable or water resistant.

In the illustrated embodiment, the tubing 114 is adjacent the inner layer 302. In the illustrated embodiment, the tubing 114 is attached to the layers by sewing the tubing 104 to the vest 102 with thread 116. In one such embodiment, a coating is applied to the inner layer 302 to seal the openings created by sewing the threads 116, thereby preventing seepage or wicking of any fluid in the vest 102 to anything inside the vest 102. In other embodiments, the tubing 114 is positioned between the layers 302, 304, 306 or adjacent the outside layer 302. An advantage of positioning the tubing 114 adjacent the inner layer 302 is that the outer layer 306 has a surface area for evaporation that is maximized and not blocked by the tubing 114 on the outer surface. Such an advantage is also realized if the tubing 114 is positioned between the inner and outer layers 302, 306, for example, in the middle layer 302 adjacent the inner layer 302.

The vest 102 is an evaporative cooling device. The inner layer 302 defines the interior of the garment 100 because it is adjacent the body of the wearer. The inner layer 302 and is a fabric, sheet, or film that prevents or inhibits fluid from the vest 102 passing or wicking to anything inside the vest 102, such as a layer that is water repellent or water resistant. A water repellent layer is one that resists but is not impervious to water. A water resistant layer is one resisting, though not entirely preventing, the penetration or passage of water. In one embodiment, the inner layer 302 is impermeable or impervious to water and water vapor.

The outer layer 306 is a porous material that freely passes water vapor during evaporation. The outer layer 306 also passes liquid water to charge the middle layer 304. The outer layer 306 has a porous region through which the liquid water and water vapor passes. The middle layer 304 includes a material that absorbs liquid water and releases it as water vapor after a phase change. The outer layer 306 is impermeable or impervious to the water absorbing material in the middle layer 304.

In one embodiment, the middle layer 304 includes a polymer embedded fabric that contains highly absorbent crystals or superabsorbent polymers. Superabsorbent polymers (SAP) (also called slush powder) are polymers that can absorb and retain extremely large amounts of a liquid relative to its own mass. In another embodiment, the middle layer 304 includes embedded polyacrylamide crystals. Polyacrylamide crystals are capable of absorbing and retaining large quantities of water compared to the weight and volume of the polyacrylamide crystals.

To charge the vest 102 for use, the garment 100 is immersed in liquid water for one or more minutes. The middle layer 304 quickly absorbs the liquid as the vest 102 charges. When the garment 100 is worn after being charged, the heat from the body wearing the garment 100 and the heat from outside sources, such as the sun, causes the middle layer 304 to release the water through evaporation. The water in the middle layer 304 evaporates using heat energy for the phase transition and thereby cooling the garment 100 and the wearer.

The rate of evaporation is slower than the uptake of water by the middle layer 304, thereby providing evaporative cooling for an extended period. The rate of evaporation determines the amount of heat removed by passive cooling and the time passive cooling is available. The rate of evaporation is determined by various factors, such as the relative humidity and the temperature differential between the inner and outer layers 302, 306. The normal temperature differential during use of the garment 100 is that the inner layer 302 has a higher temperature than the outer layer 306. The inner layer 302 is heated by the body wearing the garment 100. The outer layer 306 is cooled by evaporative cooling of the middle layer 304.

The tubes 114, with cool water flowing through them, provide active cooling for the garment 100. The cool water flowing through the tubes 114 absorb a portion of the heat from the body wearing the garment 100. The cool water flowing through the tubes 114 also lowers the temperature differential between the inner and outer layers 302, 306 by decreasing the temperature of the inner layer 302, which is also being cooled by evaporation of the water in the middle layer 304. The lower differential temperature reduces the rate of evaporation, thereby allowing the middle layer 304 to function as an evaporative cooler for a longer time. For the case where the heat load is such that the tubes 114 cannot reduce the temperature differential, the additional cooling from the tubes 114 aids in removing heat from the body wearing the garment 100 because the evaporative cooling can remove only a limited amount of heat for a specific differential temperature.

FIG. 4 illustrates a schematic diagram showing a one embodiment of multi-mode cooling system 400. The system 400 includes a multitude of garments 100-A, 100-B, 100-C, and a cool water supply 402. FIG. 4 illustrates a simplified schematic of one embodiment of a cooling system 400. The simplified schematic does not illustrate various connections, for example, isolation valves and tubing connectors between the various components; however, those skilled in the art will recognize the need for such components and understand how to construct such a system 400.

Each garment 100 includes an inlet connector 112-i and an outlet connector 112-o. The inlet connectors 112-i are connected to conduit connected to an outlet manifold 404-o of the water supply 402 and the outlet connectors 112-o are connected to a conduit connected to an inlet manifold 404-i of the water supply 402. Although only three garments 100-A, 100-B, 100-C are illustrated, the system 400 is able to use various numbers of garments 100. In one such embodiment, the manifolds 404 are appropriately sized to accommodate the number of garments 100 desired to be connected to the system 400.

The water supply 402 includes a pump 406 and a reservoir 408. The pump 406 takes suction from the reservoir 408 and supplies pressurized water to the outlet manifold 402-o. The pressurized water is then pushed through the conduits to the inlet connectors 112-i of the connected garments 100. The pressurized water circulates through the tubing 114 and discharges through the outlet connectors 112-o, where it enters the inlet manifold 404-i. The inlet manifold 404-i collects the discharges from the connected garments 100 and directs the discharges to the reservoir 408. The reservoir 408, in one embodiment, is filled with water and ice to provide chilled water to the garments 100. For example, block ice or loose ice is placed in the reservoir 408, along with a little water. The reservoir 408 is insulated such that the water returned from the garments 100 is the primary heat source that causes the ice to melt, thereby cooling the water. In one such embodiment, the pump 406 is battery operated and the water supply 402 is portable. In another embodiment, the reservoir 408 is filled with water that is chilled by a chilling unit that maintains the water at a desired temperature. In yet another embodiment, the water supply 402 provides warmed or heated water to the garments 100, which are suitable for use in a cold environment.

The garment 100 and cooling system 400 include various functions. The function of cooling passively is implemented, in one embodiment, by the vest 102 having a material that is able to receive and absorb an amount of liquid water with a mass greater than the material receiving the water. In one such embodiment, the material is a polymer that is contained within the vest 102, for example, by being embedded in a fabric or contained in a bag or pouch.

The function of cooling actively is implemented, in one embodiment, by the tubing 114 attached to the vest 102. In one such embodiment, the tubing 114 has a connector 112 attached at each end. The connectors 112 attach to a conduit that is in fluid communication with a water supply 402.

The function of supplying water to a garment 100 is implemented, in one embodiment, by a water supply 402 that contains a pump 406 and a reservoir 408. The reservoir 408 is configured to hold a volume of water.

From the foregoing description, it will be recognized by those skilled in the art that a garment 100 and cooling system 400 has been provided. It is advantageous to combine active cooling with evaporative cooling in a single, multi-mode cooling garment 100. Doing so results in a less bulky, smaller sized, and lighter weight garment 100 than either an active cooling garment or an evaporative cooling garment. The garment 100 with passive cooling working in conjunction with active cooling results in a cooling device that cools faster and for a longer time than a device with only passive or active cooling. These advantages allow the multi-mode cooling garment 100 to be more comfortable when worn for extended periods.

In one embodiment of the multi-mode cooling garment 100, the garment 100 is an evaporative cooling garment with active cooling tubing arranged within defined areas of the garment 100. The active cooling portion of the multi-mode cooling garment 100 allows the garment 100 to be less bulky and lighter in weight than an evaporative cooling garment, by itself, with the same temperature differential and duration of cooling. Likewise, the addition of evaporative cooling to an active cooling system allows the active cooling system to remain active for a longer duration and/or use less cold producing material, such as an ice block or crushed ice. Requiring less cold producing material allows the supporting equipment for the active cooling garment to be smaller in size and lighter in weight.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. An apparatus for cooling a person in extreme environments, said apparatus comprising:
a garment having a first layer and a second layer, said garment having a middle layer between said first and second layers, said first layer defining an interior of said garment, said first layer being substantially water resistant, said middle layer including a material capable of absorbing liquid water and releasing said liquid water as water vapor, said second layer being permeable such that said liquid water and said water vapor are passable through said second layer,
said garment including a fluid conduit in fluid communication with an inlet connector and an outlet connector, a portion of said fluid conduit having a serpentine pattern defining a heat transfer area on said garment;
a pump having a discharge in fluid communication with said inlet connector of said fluid conduit; and
a reservoir configured to contain a quantity of water, said reservoir in fluid communication with an intake of said pump, said reservoir in fluid communication with said outlet connector of said fluid conduit.

2. The apparatus of claim 1 wherein said garment is a vest configured and dimensioned to fit snugly about a body portion of the person.

3. The apparatus of claim 1 wherein said garment includes a front section and a back section joined with a pair of members, at least one of said pair of members being elastic, said garment configured to fit snugly about a body portion of the person.

4. The apparatus of claim 1 wherein said material is a polymer that absorbs liquid water.

5. The apparatus of claim 1 wherein said material is a polymer that absorbs liquid water, and said polymer is embedded in said middle layer.

6. The apparatus of claim 1 wherein said material is embedded in said middle layer and said first and second layers are impermeable to said material.

7. The apparatus of claim 1 further including at least one manifold configured to connect a plurality of said garments to said pump and said reservoir.

8. The apparatus of claim 1 wherein said first, middle, and second layers are joined with a quilting stitch that allows said middle layer to expand between said first and second layers when said middle layer absorbs said liquid water.

9. An apparatus for cooling a person, said apparatus comprising:
a first layer defining an interior configured to be occupied by a body portion of the person, said first layer being substantially water resistant;
a second layer being permeable such that liquid water and water vapor are passable through said second layer;
a middle layer positioned between said first and second layers, said middle layer including a material capable of absorbing liquid water; and
a fluid conduit in fluid communication with an inlet connector and an outlet connector, a portion of said fluid conduit defining a heat exchange area on said first layer.

10. The apparatus of claim 9 wherein said first layer is configured and dimensioned to fit snugly about a body portion of the person.

11. The apparatus of claim 9 wherein said fluid conduit is arranged in a serpentine pattern in said heat exchange area.

12. The apparatus of claim 9 wherein said first layer, said middle layer, and said second layer are quilted together to form a sheet.

13. The apparatus of claim 9 wherein said first, second, and middle layers are joined with a quilting stitch that allows said middle layer to expand between said first and second layers when said middle layer absorbs said liquid water.

14. The apparatus of claim 9 wherein said material is a polymer that absorbs liquid water, and said polymer is embedded in said middle layer, and said first and second layers containing said material therebetween.

15. An apparatus for cooling a person in extreme environments, said apparatus comprising:
a sheet having a first surface and a second surface, said first surface being substantially water resistant, said second surface defining a porous region permeable to liquid water and to water vapor, a material between said first and second surfaces capable of absorbing said liquid water and releasing said water vapor, at least one fluid conduit defining a heat exchange area on said sheet;
a first connector defining an inlet to said fluid conduit; and
a second connector defining an outlet to said fluid conduit, said first connector in fluid communication with said second conduit through said fluid conduit.

16. The apparatus of claim 15 wherein material is a polymer that absorbs liquid water.

17. The apparatus of claim 15 wherein said sheet is configured and dimensioned to conform to a body portion of the person.

18. The apparatus of claim 15 wherein said fluid conduit is arranged in a serpentine pattern within said heat exchange area.

19. The apparatus of claim 15 wherein said first and second surfaces are joined with a quilting stitch that allows said material to expand between said first and second surfaces when said material absorbs said liquid water.

20. The apparatus of claim 15 further including a water supply configured to connect to said first and second connectors, said water supply including a pump and a reservoir, said pump in fluid communication with said reservoir and said first connector, and said reservoir in fluid communication with said second connector.
